(19) United States
(12) Patent Application Publication
(10) Pub. No.: US 2025/0223408 A1
(43) Pub. Date: Jul. 10, 2025

(54) POROUS HYBRID MATERIAL, METHOD FOR THE PRODUCTION THEREOF AND THE USE THEREOF AS SORBENT

(71) Applicant: SCIXTEC GMBH, Offenburg (DE)

(72) Inventor: Adrian MONTELEONE, Oberkirch (DE)

(73) Assignee: SCIXTEC GMBH, Offenburg (DE)

(21) Appl. No.: 18/853,192

(22) PCT Filed: Mar. 20, 2023
(86) PCT No.: PCT/EP2023/057076

§ 371 Date: Oct. 1, 2024

(30) Foreign Application Priority Data
Apr. 12, 2022 (EP) 22166346.1

(51) Int. Cl. C08J 3/14; B01J 20/20; B01J 20/26; B01J 20/28; B01J 20/32; C02F 1/28; C02F 101/38; C08L 77/02; C09K 21/02; C09K 21/14; C12N 1/00

(52) U.S. Cl. CPC C08J 3/14; B01J 20/20; B01J 20/262; B01J 20/28019; B01J 20/28026; B01J 20/3212; B01J 20/324; C02F 1/288; C08L 77/02; C09K 21/02; C09K 21/14; C12N 1/00; C02F 1/283; C02F 1/285; C02F 2101/38; C08J 2377/02

(57) ABSTRACT

A process for producing a porous hybrid material based on plastics polymer is provided including the steps of: converting a plastics polymer or a mixture of different plastics polymers into a solution by addition of a solvent or a solvent mixture of at least two solvents; admixing one or more different functional materials; precipitating the hybrid material including the plastics polymer or the mixture having different plastics polymers and the functional material or two or more different functional materials by addition to a liquid and/or gaseous precipitant. A porous hybrid material and to the use thereof are also provided.

### CROSS-REFERENCE TO RELATED APPLICATIONS

This application is a 371 National Phase of PCT/EP2023/057076, filed Mar. 20, 2023, which claims priority from European Patent Application No. 22166346.1, filed Apr. 1, 2022, both of which are incorporated herein by reference as if fully set forth.

### TECHNICAL FIELD

The present invention relates to a process for producing a porous material. The present invention also relates to a porous hybrid material and to the use thereof.

### BACKGROUND

Every day, residues of products produced or caused by humans, also known as anthropogenic trace substances, are continuously released into the environment. Improved analytical capabilities have in recent years made it possible to determine their concentration in the air and especially in waterbodies. Although the trace substances are present in very low concentrations they have an impact on the environment and in some cases are degradable only with difficulty in natural water bodies and in the air. They can further cause damage in organisms, including humans, which is why their elimination from the water and the air is one of the great challenges of the coming years. This is especially the case when it is considered that of the approximately 50 million chemical compounds that are in circulation worldwide and can therefore form trace substances, 5000 are considered as potentially relevant to the environment.

Trace substances in waterbodies and wastewaters that can be detected in a low concentration of less than one millionth of a gram (<1 μg/1) include for example pharmaceuticals, x-ray contrast agents, fragrances in personal care and cleaning compositions, biocides, flame retardants, perfluorinated chemicals (PFC) and substances with hormone-like effects.

Their removal from wastewaters is carried out in sewage treatment plants. A modern sewage treatment plant purifies wastewater in three stages. Pretreatment includes mechanical wastewater treatment. In this first purification stage, larger solids are removed from the dirty water by mechanical apparatuses such as rakes and sand traps. Wastewater treatment is continued by way of a mechanical dewatering (screenings press) and subsequent disposal. The sand settles in the sand trap. It is then washed mechanically in the sand trap apparatus. It may then be recovered. The finer solids sink to the bottom of the pretreatment tank where they form the sewage sludge. This drains via pipes once it has reached a certain height in the tank. It is then dewatered and sent to the digester for recovery. The suspended solids are routed via overflows into the second clarifier.

During the second purification stage (biological wastewater purification) decomposition of soluble organic substances is effected by bacteria. The decomposition of fecal matter, food residues and organic dirt from washing through bacteria is possible only with introduction of oxygen. This is passed directly into the wastewater in the form of compressed air. The bacteria digest carbon to release water and CO₂. They also generate energy through decomposition of biological substances. Since they always have enough nutriment they multiply correspondingly vigorously and form the activated sludge. At this stage of wastewater treatment, only 5% to 10% of organic substances, phosphorus and nitrogen compounds remain in the pretreated water. This residue is removed from the water virtually completely in the third purification stage.

This stage comprises metabolization of nitrogen by certain bacteria types. The phosphorus is eliminated by a chemical process. Precipitation is usually effected using aluminum chloride or iron sulfate. These metal compounds make the phosphorus insoluble in water so that it settles on the bottom. It may then be sent for disposal. Only very few sewage treatment plants also have a 4th purification stage that is capable of eliminating hormones, pesticides and herbicides as well as pharmaceutical residues through the use of activated carbon. In certain cases ozone is also employed to kill pathogenic microorganisms and neutralize pharmaceuticals with subsequent activated carbon filtration.

However, investigations have shown that many trace substances are very difficult to remove from the wastewaters, with the result that sewage treatment plants require a fourth purification step.

For further removal of trace substances in sewage treatment plants by the fourth purification stage essentially two process techniques are presently available:
Ozonation: By introducing the strong oxidant ozone into the pre-purified wastewater the trace substances are largely decomposed;
Activated carbon adsorption: Trace substances can be bound to activated carbon, which is employed either as powder or in granular form (granules).

However, the fourth purification stage generates not only additional costs but also additional environmental impacts in terms of energy and equipment requirements and waste generation.

The air contains not only the natural constituents of air but also anthropogenically generated air contaminants such as for example nitrogen oxides, carbon monoxide and sulfur dioxide which can lead to long-term changes in the corresponding proportions of the respective constituents. The trace substances that are classed as greenhouse gases and are, above all, long-lived, for example methane and carbon dioxide, can also have effects on climate. The air also contains particulate matter generated by emissions from motor vehicles, power and district heating plants, furnaces and heating systems in residential buildings, in metal and steel production or else in the handling of bulk materials.

The removal of anthropogenic trace substances from the air may be carried out by mechanical, biological or thermal processes. In biological processes, gaseous trace substances are decomposed by microbiological means. However, since the ingredients must be biodegradable and can only be present in low concentrations the field of application of biological processes is very limited. Biological methods are predominantly employed in the case of odor problems.

The aim of mechanical processes is the separation of particles from an offgas stream (dedusting). The separation of fine dusts in particular is of great importance.

Thermal processes are used to remove gaseous impurities. The most commonly used processes include absorption and adsorption. Both processes are versatile and are suitable, for example, for removing nitrogen oxides, sulfur dioxide, hydrogen sulfide and carbon dioxide. It is in principle the case for both processes that the offgas should be very largely dust-free and a mechanical purification may therefore have to be carried out initially. At least three components are involved in a sorption: the pollutant to be separated, the carrier gas and a solvent. The solvent absorbs the gaseous substance, wherein this may be by physical or chemical means. To ensure that the solvent absorbs only the pollutant and not the carrier gas, the solvent must be adapted to the specific application. During adsorption the pollutant to be separated is bonded to the surface of a solid (adsorbent). As with absorption this may be effected by physical or chemical means. A very commonly employed adsorbent is activated carbon. Adsorption is facilitated by low temperatures. The offgas to be purified should thus have a temperature below 30° C.

As detailed above, it is presently activated carbon that is the focus for removal of trace substances from the air and from wastewaters. Worldwide demand is therefore very high and the total market volume was estimated to be around 8.13 billion US dollars in 2021. The European share for 2021 was about 1.85 billion US dollars. The main areas of application for activated carbon are assigned to the two sectors of air purification and water treatment, wherein 41% of the worldwide total is accounted for by air purification and a further 41% is accounted for by water treatment.

However, the activated carbon market is not environmentally friendly for a variety of reasons.

The manufacturing process is very energy-intensive and not particularly efficient. Activated carbon is produced from vegetable, petrochemical or mineral starting materials. This comprises initially producing a raw activated carbon using dehydrating agents or by dry distillation. The raw activated carbon then requires further processing in order to impart it with a largest possible surface area which can in turn interact with the largest possible spectrum of substances on account of its surface area properties. In order to impart carbon with these properties it must be partially oxidized (partially burnt). Three processes occur:
So-called condensates, i.e. carbon compounds on the carbon which are not part of the actual basic structure of the carbon, are detached (desorbed) and expelled. This opens “clogged” pores (especially mesopores in the um region) in the carbon, thus enlarging the surface area.
A more labile fraction of the carbon basic structure of the carbon is burnt, thus forming new pores (especially micropores in the nanometer range).
The surface area of the carbon basic structure is partially oxidized so that not only the aromatic basic structure of the carbon but also functional groups (carboxyls, aldehydes, ketones) influence the chemistry of the surface, thus making it hydrophilic (readily wettable with water).

To this end the raw activated carbon is heated to high temperatures (>850° C.) and treated with oxidizing gases such as air or oxygen, steam or CO₂. At these high temperatures the labile carbon fractions and condensates react according to the following reaction equations:

C+O₂→CO₂

C+H₂O→CO+H₂

C+CO₂→2 CO

Activation with these gases is also called “physical” activation because it was previously thought that the hot steam only “cleans” the carbon pores, though the process is actually based on chemical reactions. The starting carbon may be fresh or else previously carbonized biomass or else bituminous or lignitic coal. Fossil materials are often cheaper and more homogeneous.

“Chemical” activation by contrast refers to the treatment of the starting material (for example wood, coconut shells or sugar) with chemicals such as zinc chloride or phosphoric acid, wherein temperatures above 600° C. must likewise be achieved.

The process is thus not only very costly and complex but also not particularly effective. The production of one ton of activated carbon requires 3.5 to 5 tons of bituminous coal or 5 to 6.5 tons of lignitic coal. The production of one ton of coconut fiber carbon requires 10-13 tons of coconut shells. Since the energy cost of the process is so high, emissions of 11-18 tons of CO₂ equivalents per ton of activated carbon are formed in practice.

The world market is also dominated by suppliers from East Asia, thus resulting in long transport routes. Activated carbon is usually also obtained from fossil bituminous coal and is subject to fewer environmental protection regulations in these countries.

The prior art discloses attempts to minimize the use of activated carbon in air and water purification by using plastics polymer-based materials for these purposes instead of activated carbon.

WO 2021/005212 discloses a process for producing a porous material element based on a plastics polymer. The process may be used to produce elements having a (micro)porous structure which provides a large absorption surface area for many pollutants. These can then replace activated carbon.

However, these materials have the disadvantage that they do not allow targeted and selective purification of the trace substances. This is because the materials disclosed in WO2021/005212 consist exclusively of one material. They therefore exhibit exclusively the properties specified by this material. Scope for functionalization, for example sulfonation or nitration or porosity alteration of such materials is also limited. It is also known that the sorption performance of such monomaterials is not as effective.

### SUMMARY

In view of the above it is an object of the present invention to provide novel materials which have a better CO₂ balance than activated carbon and allow selective removal of trace substances on account of their variability while simultaneously being economic.

This object is achieved by a process having one or more of the disclosed herein. Advantageous embodiments of this process may be found in the description and claims that follow.

The object of the present invention is further achieved by the porous hybrid material and by the use of the porous hybrid material.

The process according to the invention for producing a porous hybrid material comprises the steps of:
a) converting a plastics polymer or a mixture comprising different plastics polymers into a solution by addition of a solvent or a solvent mixture of at least two solvents, wherein the solvent is selected from the group comprising:
inorganic acids or a mixture of different inorganic acids selected from the group comprising: sulfuric acid, nitric acid, hydrochloric acid, phosphoric acid, hydrohalic acids, hydrogen peroxide;
organic acids or a mixture of different organic acids selected from the group comprising: formic acid, acetic acid;
organic hydrocarbon solvents selected from the group comprising: THF, toluene, decalin;
b) admixing one or more different functional materials, wherein the functional materials are selected from the list consisting of:
   0.1% to 40% by weight of carbon-based ab- and adsorbents,
   2% to 40% by weight of polymeric or plastic-containing ab- and adsorbents,
   2% to 40% by weight of oxidic ab- and adsorbents,
   2% to 40% by weight of ab- and adsorbents of biological origin selected from the group comprising: substances containing cellulose, starch, protein, chitin, chitosan,
   2% to 50% by weight of luminescent substances,
   0.1% to 30% by weight of indicator substances (e.g. color indicator for pH);
c) precipitating the hybrid material comprising the plastics polymer or the mixture comprising different plastics polymers and the functional material or two or more different functional materials by addition to a liquid and/or gaseous precipitant.

In the context of the present invention the term “hybrid material” is to be understood as meaning a material comprising at least two or more constituents. The constituents are chemically or physically bonded to one another.

In the context of the present invention the term “plastics polymer” is to be understood as meaning any compound composed of at least two or more monomers of preferably synthetic and/or partly synthetic origin. The term likewise refers to reinforced polymers, for example glass fiber- or carbon fiber-reinforced polymers.

In the context of the present invention the term “polyamides” (PA for short) is to be understood as meaning linear polymers with regularly repeating amide bonds (—CO—NH—) along the main chain which are produced by polycondensation as autocondensate (PAX-X corresponds to the length of the carbon backbone, for example PA6) or as co-condensate (PAXY: X corresponds to length of the carbon backbone in the diamine; Y corresponds to length of the carbon backbone in the diacid, for example PA6.10).

Suitable monomers for polyamides particularly include aminocarboxylic acids, lactams and/or diamines and dicarboxylic acids. Polyamides may be arranged into the following classes according to the type of monomers:
aliphatic polyamides: the monomers derive from aliphatic basic structures, for example PA from caprolactam (PA 6: explanation of abbreviations see below) or from hexamethylenediamine and adipic acid (PA 66);
semiaromatic polyamides: the monomers are derived in part from aromatic basic structures, for example PA from hexamethylenediamine and terephthalic acid (PA 6T);
aromatic polyamides (polyaramides): the monomers are derived from purely aromatic basic structures, for example para-phenylenediamine and terephthalic acid (aramid).

In the context of the present invention the term “polyethylene” (abbreviation PE) is to be understood as meaning a polymer produced by polymerization of ethylene and having the chain structure formula [—CH₂—CH₂—]n. A distinction is made between: PE-HD (low-branched, high-density polymer chains), PE-LD (high-branched, low-density polymer chains), PE-LLD (low-density linear polyethylene whose polymer molecule comprises only short branches), PEHMW (high-molecular-weight polyethylene), and PE-UHMW (ultrahigh-molecular-weight polyethylene having an average molar mass of up to 6000 kg/mol).

In the context of the present invention the term “polypropylene” (abbreviated PP) is to be understood as meaning a plastic produced by polymerization of propene. Polypropylene can be divided into atactic polypropylene, syndiotactic polypropylene and isotactic polypropylene. In atactic polypropylene the methyl group is randomly aligned, in syndiotactic polypropylene it is alternatingly aligned and in isotactic polypropylene it is uniformly aligned on one side of the macromolecular carbon backbone.

In the context of the present invention, the term “polystyrene” (PS for short) is to be understood as meaning a polymer produced by polymerization of the monomer styrene. The asymmetry of the styrene molecule results in different possible orientations of the monomer units in the polymeric molecule. They are determined by the respective conditions during the polymerization. A distinction is made between isotactic, syndiotactic and atactic polymers. The isotactic styrene polymer where all monomer units are spatially unidirectional has not acquired any industrial significance. By contrast, the amorphous, atactic styrene polymer, whose monomer units occupy random spatial positions is the most important polystyrene plastic which is predominantly used to produce mass-produced products.

In the context of the present invention, the term “polyurethane” (PUR for short) is to be understood as referring to plastics or synthetic resins formed from the polyaddition reaction of dialcohols (diols) or polyols with polyisocyanates. The urethane group (—HN—CO—O—) is characteristic of polyurethanes.

In the context of the present invention the term “polyvinyl chloride” (PVC for short) is to be understood as meaning a thermoplastic polymer produced from the monomer vinyl chloride by chain polymerization.

In the context of the present invention the term “copolymer” is to be understood as meaning polymers composed of at least two types of monomer units. Such bi- or multifunctional copolymers can be divided into five classes: statistical copolymers in which the distribution of the two monomers in the chain is random; gradient copolymers, in principle similar to the statistical copolymers but with a variable proportion of a monomer over the chain; alternating copolymers having a recurring arrangement of the monomers along the chain; block copolymers consisting of two longer sequences or blocks of each monomer; and graft copolymers where blocks of one monomer are grafted onto the backbone of another monomer. In the context of the present invention the term is likewise to be understood as meaning polymer blends composed of two or more polymers.

In the context of the present invention the term “functional materials” is to be understood as meaning all additives which are added to the plastics polymer to produce the porous hybrid material according to the invention.

In the context of the present invention the term “adsorbents” is to be understood as meaning chemical substances on whose surface other substances adhere. The adhesion is generated by physical attractive forces such as for example Van der Waals forces, dipole-dipole interactions, π-π interactions and also hydrogen bonds and ionic interactions.

In the context of the present invention the term “absorbents” is to be understood as meaning chemical substances where another substance diffuses into the interior.

In the context of the present invention the term “carbon-based ab- and adsorbents” is to be understood as meaning but is not limited to: activated carbon, activated coke, carbon molecular sieves, graphites, graphenes.

In the context of the present invention the term “polymeric or plastics-containing ab- and adsorbents” is to be understood as meaning but is not limited to: polyamides, polystyrene, polypropylene, polyethylene, polyethylene terephthalate, polyvinyl chloride, acrylonitrile-butadiene-styrene copolymers.

In the context of the present invention the term “oxidic ab- and adsorbents” is to be understood as meaning but is not limited to: silicates, silica gel, activated alumina, talc, zeolite, aluminum oxide, titanium dioxide.

In the context of the present invention the term “biological ab- and adsorbents” is to be understood as meaning compounds and their derivatives of biological origin including but not limited to: substances containing cellulose, starch, protein, chitin, chitosan.

In the context of the present invention the term “luminescent substances” is to be understood as meaning substances that emit light by excitation with energy in the form of chemical energy, electrical energy or UV light.

In the context of the present invention the term “indicator substances” is to be understood as meaning but is not limited to: color indicator for pH.

In the context of the present invention the term “precipitant” is to be understood as meaning an agent or a mixture of different agents that is intended to bring about a physical, chemical or physico-chemical reaction where a dissolved substance reacts to form an insoluble solid.

The process according to the invention has the considerable advantage that it allows rapid adaptation of the composition of the hybrid material for novel applications.

The porous hybrid materials obtained by the process according to the invention have the considerable advantage over the monomaterials known from the prior art that they are characterized by a high variability and flexibility in possible applications. Through selection of the functional material the hybrid material may be produced specifically adapted to its end application. For example the hybrid materials according to the invention may be produced such that they absorb one or more trace substances very selectively. This selectivity is becoming ever more important, especially in applications in the field of medicine or food technology.

Furthermore, flexibility in adapting such a hybrid material to new trace substances is of great importance, especially in view of the continuously increasing number of trace substances to be found in wastewaters and in the air. In the case of the hybrid materials according to the invention this will be done simply through exchange of the respective functional material.

The hybrid material obtained by the process according to the invention moreover has a much better CO₂ balance than activated carbon. As specified in detail above activated carbon is very costly and complex to produce and production and activation thereof require a great deal of energy, thus resulting in very high CO₂ emissions. The production of the hybrid material according to the present invention is considerably more environmentally friendly. This results from the fact that for most applications of the hybrid material plastics wastes and/or recycled products are used as starting materials, even plastics wastes and/or recycled products that have not previously been subjected to cleaning.

This significantly enhances the environmental compatibility of the hybrid materials according to the invention.

It is only for applications subject to extremely stringent requirements, such as in the medical sector, where no plastics wastes and/or recycled products are used as starting materials. However, even when virgin plastics polymers are used, the production thereof is not as energy intensive as production of activated carbon.

Furthermore, due to the polymer matrix the hybrid materials according to the invention are characterized by a higher calorific value upon incineration (such as in waste incineration plants) or pyrolyses compared to activated carbon. This can also have a positive effect on the energy balance.

In one embodiment of the invention the solvent is an acid selected from the group consisting of: hydrochloric acid, sulfuric acid, nitric acid, formic acid, acetic acid, preferably hydrochloric acid and sulfuric acid.

In a further embodiment of the invention the solvent is an organic solvent selected from the group comprising: THF, toluene, decalin.

In one embodiment of the invention the solvent is a mixture comprising sulfuric acid and hydrogen peroxide in a ratio of 4 to 1.

In one embodiment of the invention the solvent is a mixture comprising 96% sulfuric acid and 30% hydrogen peroxide in a ratio of 4 to 1.

In a further embodiment of the invention the concentration of the aqueous acid is at least 10% and at most 99%. In a preferred embodiment the concentration is 16% to 96%.

The selection of the solvent or of the mixture comprising different solvents must be made in such a way that this also generates separation steps. This is particularly important when using uncleaned and/or unsorted waste as the starting material.

In a further embodiment of the invention the ratio of the plastics polymer or the mixture comprising different plastics polymers to the solvent is from 5:100 to 60:100. In detail the ratio of the plastics polymer or the mixture comprising different plastics polymers is from 5 g per 100 g of the solvent to 60 g of the plastics polymer or the mixture comprising different plastics polymers per 100 g of the solvent.

The admixing of one or more different functional materials may be carried out in different ways. Only two options are described here by way of example. However, the invention is not limited to these. The admixing of the functional material or the functional materials may be performed after step a) through incorporation by stirring or kneading depending on the viscosity of the solution present. The admixing may likewise be carried out during step a).

In a further embodiment of the invention the amount of the functional material or of the mixture is 0.1% to 60% by weight, preferably 0.2% to 45% by weight, more preferably 0.5% to 25% by weight. In a further embodiment the amount of the one or more different functional materials is 10% to 20% by weight, preferably 10% to 15% by weight.

The precipitation of the hybrid material according to step c) may be carried out for example such that the polymer additive solution is transferred into a precipitant and thus precipitates.

In one embodiment the precipitant is selected from the list comprising: water, acids, bases and mixtures thereof. The precipitant may be in liquid form or in vaporous form.

The precipitant is preferably water. This has the advantage that the process according to the invention is exceptionally unconcerning and cost-effective.

The precipitant is more preferably selected from the list comprising aqueous solutions of NaOH, Ca(OH)₂, KOH, HCl and H₂SO₄.

In one embodiment the precipitation is achieved by addition to a liquid and/or gaseous precipitant. In this embodiment the solution obtained in step b) is added to the vessel comprising the precipitant by dropwise addition in portions, pouring, stirring, immersing or spraying (e.g. with nozzles). If the precipitant is gaseous the addition is carried out in a chamber for contacting with vapor/gas. In this case the gaseous precipitant may already be present in a defined concentration or may be iteratively adjusted to the desired concentration (saturation). The duration of incubation in the gaseous precipitant depends on different parameters (e.g. thickness of the material, temperature, material form, hydrophobicity/hydrophilicity, porosity).

In a further embodiment step b) is followed by contacting of the solution comprising the plastics polymer or the mixture comprising different plastics polymers and the functional material or two or more functional materials with an inert gas or foaming using substances capable of gas formation. This additional step results in an open-cell and/or closed-cell or mixed-cell structure to achieve an increase in volume of the material and thus reduce weight. The solution may be contacted with an inert gas so that this gas is intensively distributed in the composition (with mixing processes). When this contacting with gas is carried out in a suitable pressure-stable container an increase in volume is achieved, for example into a precipitant-permeable form (e.g. cylinder) if the pressure is maintained. The introducing of a liquid or gaseous precipitant (e.g. water or steam) into the pressure container to the foamed composition causes the foam to be stabilized in the precipitant-permeable form (e.g. semipermeable membrane) as a result of the slow phase separation of the solvent and precipitant and it may be withdrawn after a time. When using a substance capable of gas formation or a coated substance capable of gas formation (delayed release) the composition may likewise be foamed and be stabilized using a liquid or gaseous precipitant by the initiated phase separation.

In one embodiment the porous hybrid material comprises 0.1% to 60% by weight, preferably 0.2% to 45% by weight, more preferably 0.5% to 25% by weight, of the one or more different functional materials. In one embodiment the porous hybrid material comprises 10% to 20% by weight, preferably 10% to 15% by weight, of the one or more different functional materials. This measure has the considerable advantage that the porous hybrid material is cost-effective but at the same time shows improved effectiveness as demonstrated by the tests.

In a further embodiment the plastics polymer is polyamide 6, polyamide 6.6 or polyamide 12, particularly preferably polyamide 6.

In a further embodiment the plastics polymer is made of plastics wastes and/or recycled products.

In a further embodiment the plastics polymer is made of plastics wastes and/or recycled products that have not been subjected to a preceding cleaning.

In a further embodiment the functional material is selected from the group consisting of: activated carbon, activated coke, carbon molecular sieves, graphites, graphenes, preferably from activated carbon, carbon molecular sieves and graphenes and most preferably activated carbon.

In a further embodiment the functional material is selected from the group consisting of: polystyrene, polypropylene, polyethylene, polyethylene terephthalate, acrylonitrile-butadiene-styrene copolymers, preferably from polystyrene, polypropylene, polyethylene, acrylonitrilebutadiene-styrene copolymers, most preferably polystyrene and acrylonitrile-butadiene-styrene copolymers.

In a further embodiment the functional material is selected from the group consisting of: silicates, silica gel, activated alumina, talc, zeolite, aluminum oxide, titanium dioxide, preferably from silicates, silica gel, zeolite and titanium dioxide, most preferably silicates and titanium dioxide.

The use of titanium dioxide has the considerable advantage that such hybrid materials according to the invention have a photocatalytic property and thus allow usability of light, in particular sunlight, for increasing pollutant and trace substance elimination.

In a further embodiment the functional material is selected from the group consisting of: cellulose, chitin, chitosan, preferably from cellulose and chitin.

In a further embodiment the functional material is a luminescent substance, for example strontium aluminate.

In a further embodiment the functional material is a color indicator for pH.

It is also in accordance with the present invention that the respective groups of the different functional materials may be arranged together in one hybrid material.

In one embodiment of the invention the distribution of the functional material or of the functional materials is approximately homogeneous. This means that the materials are uniformly distributed in the end product.

In a further embodiment the hybrid material according to the invention is in the form of granules, drops, balls, membranes, foams or filaments.

The size of the granules is between 0.1 mm to 20 mm, preferably 0.2 mm to 5 mm. They may be in the form of drops or balls.

The granule form has the advantage that it has the flowability of bulk materials such as sand or gravel and is thus just as easy to transport as these.

In the context of the invention “membrane” is to be understood as meaning polymer structures whose thickness is very low relative to their areal extent.

In the context of the present invention the term “foams” is to be understood as meaning polymer structures whose structure is formed by many cells (cavities, pores enclosed by the base material).

In the context of the present invention the term “filaments” is to be understood as meaning polymer structures consisting of endless fibers which form a flexible fabric.

In a further embodiment the porous hybrid material comprises pores having a size of 0.001 μm to 3000 μm. It is preferable when the pores have a size of 0.001 μm to 200 μm. It is more preferable when the pores have a size of 0.001 μm to 50 μm. It is even more preferable when the pores have a size of 0.001 μm to 20 μm. It is most preferable when the pores have a size of 0.001 μm to 10 μm. The method of measurement for the specific surface area is gas adsorption according to DIN-ISO 9277 (or DIN 66131) according to the Brunauer-Emmett-Teller method (BET method).

In one embodiment of the invention the porous hybrid material is completely openpored. In a further embodiment of the invention the porous hybrid material is partially open-pored. In the context of the present invention this means that some pores of the hybrid material are open and some are closed. In the case of the foams according to the invention reference is made to cells instead of pores (open-cell or mixed-cell foams).

In one embodiment of the invention the porous hybrid material comprises the following:
a) at least 40% by weight of a plastics polymer or a mixture comprising different plastics polymers selected from the group consisting of polyamides, polyethylene, polypropylene, polystyrene, polyurethane, polyvinyl chloride and copolymers thereof; and
b) [symbol]
   0.1% to 40% by weight of carbon-based ab- and adsorbents and/or
   2% to 40% by weight of polymeric or plastics-containing ab- and adsorbents and/or
   2% to 40% by weight of oxidic ab- and adsorbents and/or
   2% to 40% by weight of ab- and adsorbents of biological origin selected from the group comprising: substances containing cellulose, starch, protein, chitin, chitosan; and/or
   2% to 50% by weight of luminescent substances, and/or
   0.1% to 30% by weight of indicator substances (e.g. color indicator for pH).

In one embodiment of the invention the porous hybrid material comprises the following:
a) at least 40% by weight of a polyamide; and
b) [symbol]
   0.1% to 40% by weight of carbon-based ab- and adsorbents and/or
   2% to 40% by weight of polymeric or plastics-containing ab- and adsorbents and/or
   2% to 40% by weight of oxidic ab- and adsorbents and/or
   2% to 40% by weight of ab- and adsorbents of biological origin selected from the group comprising: substances containing cellulose, starch, protein, chitin, chitosan; and/or
   2% to 50% by weight of luminescent substances, and/or
   0.1% to 30% by weight of indicator substances (e.g. color indicator for pH). In one embodiment of the invention the porous hybrid material comprises the following:
a) at least 40% by weight of PA6, PA66, PA12; and
b) [symbol]
   0.1% to 40% by weight of carbon-based ab- and adsorbents and/or
   2% to 40% by weight of polymeric or plastics-containing ab- and adsorbents and/or
   2% to 40% by weight of oxidic ab- and adsorbents and/or
   2% to 40% by weight of ab- and adsorbents of biological origin selected from the group comprising: substances containing cellulose, starch, protein, chitin, chitosan; and/or
   2% to 50% by weight of luminescent substances, and/or
   0.1% to 30% by weight of indicator substances (e.g. color indicator for pH).

In one embodiment of the invention the porous hybrid material comprises the following:
a) at least 40% by weight of a polyamide,
b) 2% to 40% by weight of polymeric or plastics-containing ab- and adsorbents.

In one embodiment of the invention the porous hybrid material comprises the following:
a) at least 40% by weight of PA6, PA66, PA12;
b) 2% to 40% by weight of polymeric or plastics-containing ab- and adsorbents.

In one embodiment of the invention the porous hybrid material comprises 0.1% to 20% by weight of graphene as adsorbents b).

In one embodiment of the invention the porous hybrid material comprises 5% to 40% by weight of activated carbon as adsorbents b).

The hybrid material according to the invention may be provided for the following applications but is not limited thereto:
as a sorbent of trace substances in water and air purification;
as a growth and nutrient matrix for microorganisms;
as a filling and insulating material due to its flame retardant potential;
as a replacement material for weight reduction, for example in the automotive sector or in the aviation industry for lightweight construction. The use as a sorbent of trace substances in water and air purification was described in detail at the outset. When used as a growth and nutrient matrix for microorganisms the hybrid materials according to the invention are produced by the process in a suitable form (membrane or balls) for the application. A phosphorescent substance (strontium aluminate) is added as a functional material for production as a hybrid. These particles are then added to an illuminated bioreactor and colonized by the targeted microorganisms such as (micro) algae for example. The hybrid materials according to the invention serve as a surface area enlargement and growth matrix for the microorganisms in the reactor. A suitable illumination of the complete bioreactor is complex and the incident light cannot be completely utilized. The phosphorescent substances in the adsorbers store excess light and can release it back into the environment over a certain timeframe to supply the microorganisms. This makes it possible to develop energy-saving processes (intermittent irradiation) for the bioreactor which makes it possible to maintain active metabolism of the microorganisms, such as (micro) algae, over a longer timeframe. Specific compositions of hybrid materials according to the invention make it possible to integrate further advantages for the applications, such as for example media-stabilizing substances or selective substances for ad- and/or absorption of toxic metabolites from the microorganisms.

When used as a filling and insulating material on account of its flame-retardant potential or as a replacement material for weight reduction, for example in the automotive sector or in the aviation industry for lightweight construction, the hybrid materials are produced by the process according to the invention. The open- and/or closed-pore or mixed-pore foams may be used for thermal insulation in buildings for example. The open-pore structure also makes it possible to achieve gas and steam exchange. The polymer foams may also be used as sound or footfall/impact insulation. The porous structure also results in a weight reduction of the material and this may be advantageous in the automotive industry and in the aerospace industry.

Further advantageous properties of the invention shall now be more particularly described and elucidated with reference to examples and experiments.

### LIST OF ABBREVIATIONS

| Abbreviation | Meaning |
|---|---|
| APA6 | Activated polyamide 6 - pure processed material |
| | PA6 (mono-material) |
| APA12 | Activated polyamide 12 - pure processed material |
| | PA12 (mono-material) |
| APAK6 | Activated polyamide 6 carbon - hybrid material |
| | having a mass fraction of activated carbon |
| APAK12 | Activated polyamide 12 carbon - hybrid material |
| | having a mass fraction of activated carbon |

### EXAMPLE 1: PRODUCTION OF MONOMATERIAL APA6

APA6 was produced by the process disclosed in WO2021005212, wherein 48 g of polyamide 6 and 100 ml of 20% hydrochloric acid were employed.

### EXAMPLE 2: PRODUCTION OF HYBRID MATERIAL APAK6

For production of the APAK6 material 100 ml of a 20% hydrochloric acid were initially charged in a beaker. Subsequently 43.2 g of PA6 (polyamide 6), which are produced as swarf from a material-removing machining operation for plastics and metals, were weighed out and added to the 20% hydrochloric acid under standard conditions with manual stirring with a Teflon rod, thus forming a polymer solution. It was especially found that the provided plastics polymer still contained stainless steel and aluminum swarf but this was readily separable in a later step by density separation. After complete dissolution of the PA6 waste material 4.8 g of a powdered activated carbon was admixed as a functional material with stirring to obtain 48 g of a material mixture composed of 90% by weight polymer fraction and 10% by weight functional materials. The solution was stirred until the activated carbon appeared evenly distributed in the polymer solution. The polymer solution was transferred into another beaker through a net (0.2 mm mesh width) made of PP (polypropylene) to separate the aluminum and metal swarf. In a next step a 3000 ml measuring cup used as a reservoir vessel was filled with 2500 ml of precipitant (tap water at about 14° C.). Using a magnetic stirrer the tap water was set into rotation on a magnetic stirrer plate at about 700 rpm until a vortex was formed. The polymer solution admixed with functional material was taken up in a 10 ml pasteur pipette and added dropwise to the tap water at a distance of about 12 cm above the fill level and thus precipitated. The resulting granules (APAK6) have a stable and consistently porous structure in which the particles of powdered activated carbon are uniformly firmly embedded in a porous polymer matrix. Since the precipitant is brought into the acidic pH range with increasing proportion of the acidic polymer solution, neutralization with an alkali should be undertaken according to the amount of the polymer solution and the spheres subsequently separated by sieving. The granules separated by sieving are freed of acid and salt residues by washing with tap water in a further measuring cup and then once again separated by sieving and dried in ambient air.

### EXAMPLE 3: PRODUCTION OF MONOMATERIAL APA12

APA12 was produced by the process disclosed in WO2021005212, wherein 16 g of polyamide 12 and 100 ml of 96% sulfuric acid were employed.

### EXAMPLE 4: PRODUCTION OF HYBRID MATERIAL APAK12

For production of the APAK12 material 100 ml of a 96% sulfuric acid were initially charged in a beaker. Subsequently 14.4 g of PA12 (polyamide 12) which are generated as sprue pieces by an injection molder were weighed out and added to the 96% sulfuric acid under standard conditions with manual stirring with a Teflon rod, thus forming a polymer solution. It was especially found that the provided plastics polymer also contained metal parts such as for example nuts and bolts but these were readily separable in a later step by density separation. After complete dissolution of the PA12 waste material 1.6 g of a powdered activated carbon was admixed as a functional material with stirring to obtain 16 g of a material mixture composed of 90% by weight polymer fraction and 10% by weight functional materials. The solution was stirred until the activated carbon appeared evenly distributed in the polymer solution. The polymer solution was transferred into another beaker through a net (0.2 mm mesh width) made of PP (polypropylene) to separate the metal parts. In a next step a 3000 ml measuring cup used as a reservoir vessel was filled with 2500 ml of precipitant (tap water at about 14° C.). Using a magnetic stirrer the tap water was set into rotation on a magnetic stirrer plate at about 700 rpm until a vortex was formed. The polymer solution admixed with functional material was taken up in a 10 ml pasteur pipette and added dropwise to the tap water at a distance of about 12 cm above the fill level and thus precipitated. The resulting granules (APAK12) have a stable and consistently porous structure in which the particles of powdered activated carbon are uniformly firmly embedded in a porous polymer matrix. Since the precipitant is brought into the acidic pH range with increasing proportion of the acidic polymer solution, neutralization with an alkali should be undertaken according to the amount of the polymer solution and the spheres subsequently separated by sieving. The granules separated by sieving are freed of acid and salt residues by washing with tap water in a further measuring cup and then once again separated by sieving and dried in ambient air.

### EXAMPLE 5: PRODUCTION OF HYBRID MATERIAL APA6/BENTONITE

For production of the APA6/bentonite material 100 ml of a 44% sulfuric acid were initially charged in a beaker. Subsequently 44 g of PA6 (polyamide 6), which are produced as swarf from a material-removing machining operation for plastics and metals, were weighed out and added to the 44% sulfuric acid under standard conditions with manual stirring with a Teflon rod, thus forming a viscous polymer solution. It was especially found that the provided plastics polymer still contained stainless steel and aluminum swarf but this was readily separable in a later step by density separation. After complete dissolution of the PA6 waste material 4.89 g of a bentonite powder was admixed as a functional material with stirring to obtain 48.89 g of a material mixture composed of 90% by weight polymer fraction and 10% by weight functional materials. The solution was stirred until the bentonite appeared evenly distributed in the polymer solution. In a next step a 3000 ml measuring cup used as a reservoir vessel was filled with 2500 ml of precipitant (tap water at about 18° C.). Using a magnetic stirrer the tap water was set into rotation on a magnetic stirrer plate at about 700 rpm until a vortex was formed. The polymer solution admixed with functional material was taken up in a 10 ml pasteur pipette and added dropwise to the tap water at a distance of about 12 cm above the fill level and thus precipitated. The resulting granules (APA6/bentonite) have a stable and consistently porous structure in which the particles of bentonite are uniformly firmly embedded in a porous polymer matrix. The reaction with the 44% sulfuric acid gives the bentonite an even more porous structure. This makes it possible to increase the specific surface area of the plastics granules. Since the precipitant is brought into the acidic pH range with increasing proportion of the acidic polymer solution, neutralization with an alkali should be undertaken according to the amount of the polymer solution and the spheres subsequently separated by sieving. The granules separated by sieving are freed of acid and salt residues by washing with tap water in a further measuring cup and then once again separated by sieving and dried in ambient air.

### EXAMPLE 6: PRODUCTION OF POROUS ABS PARTICLES WITH HYDRATED LIME

For production of the porous ABS granules 50 ml of acetone were initially charged in a beaker. Subsequently 17.5 g of ABS regrind was weighed out and admixed with the acetone under standard conditions with manual stirring with a Teflon rod, thus forming a viscous polymer solution. After complete dissolution of the ABS regrind 1.94 g of a hydrated lime powder was admixed as a functional material with stirring to obtain 19.44 g of a material mixture composed of 90% by weight polymer fraction and 10% by weight functional materials. The solution was stirred until the hydrated lime appeared evenly distributed in the polymer solution. In a next step a 1000 ml measuring cup used as a reservoir vessel was filled with 800 ml of precipitant (tap water and ethanol in a ratio of 6 to 4 at about 20° C.). Using a magnetic stirrer the precipitant was set into rotation on a magnetic stirrer plate at about 700 rpm until a vortex was formed. The polymer solution admixed with functional material was taken up in a 10 ml pasteur pipette and added dropwise to the tap water at a distance of about 8 cm above the fill level and thus precipitated. The resulting granules (ABS/hydrated lime) have a stable and consistently porous structure in which the particles of hydrated lime are uniformly firmly embedded in a porous polymer matrix. The granules separated by sieving are largely freed of precipitant residues by washing with tap water in a further measuring cup. The granules are then washed in a 20% hydrochloric acid solution to convert the hydrated lime into calcium chloride which is easily soluble in water. Finally, the granules are washed again in water to dissolve the calcium chloride from the porous structure. This generates an enlarged specific surface area of the particles.

### Experiment 1: Adsorption Tests

Respective 120 ml portions of a synthetic wastewater (according to DIN EN ISO 11733) having the initial concentrations specified in table 1 were treated with 2.5 g of each of the four adsorbents (APA12, APAK12, APA6 and APAK6) for 10 hours at room temperature. The adsorbents were poured as a fixed bed reactor in glass chromatography columns having a volume of 35 ml. The synthetic wastewater was recirculated through the fixed bed reactors at a volume flow of 6 ml/min using a peristaltic pump. Trace analysis was performed by LC-MS/MS.

TABLE 1
Comparison of elimination performance between
APA12 and APAK12 and also APA6 and APAK6.

| | | APAK12 | APA12 | APAK6 | APA6 |
|---|---|---|---|---|---|
| | Initial | (residual | (residual | (residual | (residual |
| | concentration | concentration | concentration | concentration | concentration |
| Substance | in μg/l | in μg/l) | in μg/l) | in μg/l) | in μg/l) |
| 1H-benzotria- | 81 | 5.3 | 38 | 9.2 | 54 |
| zole | | | | | |
| 4-methylben- | 82 | 3.2 | 21 | 5.7 | 41 |
| zotriazole | | | | | |
| 5-methylben- | 84 | 2.9 | 19 | 4.7 | 41 |
| zotriazole | | | | | |
| Carbamaze- | 78 | 2.9 | 51 | 8.9 | 54 |
| pine | | | | | |
| Metoprolol | 60 | 0.92 | 56 | 20 | 60 |

As is clearly apparent from table 1 the adsorption performance of the hybrid materials APAK6 and APAK 12 is in all cases significantly higher than the adsorption performance of the corresponding monomaterials APA6 and APA12. The residual concentration of the respective trace elements is considerably lower for the inventive hybrid materials, in extreme cases by a factor of 60.

TABLE 2
Comparison of percentage elimination performance
between APA12 and APAK12 and also APA6 and APAK6.

| | Initial | APAK12 | APA12 | APAK6 | APA6 |
|---|---|---|---|---|---|
| | concentration | (Elimination | (Elimination | (Elimination | (Elimination |
| Substance | in % | in %) | in %) | in %) | in %) |
| 1H-benzotria- | 100 | 93.46 | 53.09 | 88.64 | 33.33 |
| zole | | | | | |
| 4-methylben- | 100 | 96.10 | 74.39 | 93.05 | 50.00 |
| zotriazole | | | | | |
| 5-methylben- | 100 | 96.55 | 77.38 | 94.40 | 51.19 |
| zotriazole | | | | | |
| Carbamaze- | 100 | 96.28 | 34.62 | 88.59 | 30.77 |
| pine | | | | | |
| Metoprolol | 100 | 98.47 | 6.67 | 66.67 | 0.00 |

Table 2 shows the results of Table 1 but in terms of elimination in %. It is likewise clear that the inventive hybrid materials show a markedly higher degree of elimination.

1. A process for producing a porous hybrid material based on plastics polymer, the process comprising:
a) converting a plastics polymer or a mixture comprising different plastics polymers into a solution by addition of a solvent or a solvent mixture of at least two solvents, wherein the solvent is selected from the group comprising:
inorganic acids or a mixture of different inorganic acids selected from the group comprising: sulfuric acid, nitric acid, hydrochloric acid, phosphoric acid, hydrohalic acids, and hydrogen peroxide;
organic acids or a mixture of different organic acids selected from the group comprising: formic acid, or acetic acid;
organic hydrocarbon solvents selected from the group comprising: THF, toluene, or decalin;
b) admixing one or more different functional materials, wherein the functional materials are selected from the group consisting of:
0.1% to 40% by weight of carbon-based ab- and adsorbents,
2% to 40% by weight of polymeric or plastic-containing ab- and adsorbents,
2% to 40% by weight of oxidic ab- and adsorbents,
2% to 40% by weight of ab- and adsorbents of biological origin selected from the group comprising: substances containing cellulose, starch, protein, chitin, or chitosan,
2% to 50% by weight of luminescent substances, and
0.1% to 30% by weight of indicator substances (e.g. color indicator for pH);
and
c) precipitating the hybrid material comprising the plastics polymer or the mixture comprising different plastics polymers and the functional material or two or more different functional materials by addition to at least one of a liquid or gaseous precipitant.

2. The process as claimed in claim 1, wherein the functional material is a carbon-based ad- or absorbent comprising at least one of carbon, activated carbon, activated coke, graphite, or graphene.

3. The process as claimed in claim 1, wherein the functional material is an oxidic ad- or absorbent.

4. The process as claimed in claim 1, wherein the functional material is a polymeric or plastics-containing ab- or adsorbent.

5. The process as claimed in claim 1, wherein the solvent is an inorganic acid or a mixture of different inorganic acids selected from the group comprising: sulfuric acid, nitric acid, hydrochloric acid, and hydrogen halide acids.

6. The process as claimed in claim 1, further comprising step b) is followed by contacting the solution comprising the plastics polymer or the mixture comprising different plastics polymers and the functional material or two or more functional materials with an inert gas or foaming using substances capable of gas formation.

7. A porous hybrid material, comprising:
a) at least 40% by weight of a plastics polymer or a mixture comprising different plastics polymers selected from the group consisting of polyamides, polyethylene, polypropylene, polystyrene, polyurethane, polyvinyl chloride and copolymers thereof; and
b) at least one of:
0.1% to 40% by weight of carbon-based ab- and adsorbents;
2% to 40% by weight of polymeric or plastics-containing ab- and adsorbents;
2% to 40% by weight of oxidic ab- and adsorbents;
2% to 40% by weight of ab- and adsorbents of biological origin selected from the group comprising: substances containing cellulose, starch, protein, chitin, chitosan;
2% to 50% by weight of luminescent substances; or
0.1% to 30% by weight of indicator substances.

8. The porous hybrid material as claimed in claim 7, wherein the material comprises:
a) at least 40% by weight of the polyamide.

9. The porous hybrid material as claimed in claim 8, wherein the material comprises:
a) at least 40% by weight of the polyamide which is at least one of PA6, PA66, or PA12.

10. The porous hybrid material as claimed in claim 7, wherein the material comprises:
a) at least 40% by weight of the polyamide, and
b) the 2% to 40% by weight of polymeric or plastics-containing ab- and adsorbents.

11. The porous hybrid material as claimed in claim 10, wherein the material comprises:
a) the at least 40% by weight of the polyamide which is at least one of PA6, PA66, or PA12.

12. The porous hybrid material as claimed in claim 7, wherein the material comprises 0.1% to 20% by weight of the carbon-based ad- or absorbent which comprises graphene as adsorbents.

13. The porous hybrid material as claimed in claim 7, wherein the material comprises 5% to 40% by weight of the carbon-based ad- or absorbent which comprises activated carbon as adsorbents.

14. The porous hybrid material as claimed in claim 7, wherein the material as an ad- and absorbent for trace substances in water and air purification.

15. The porous hybrid material as claimed in claim 7, wherein the material is a growth and nutrient matrix for microorganisms.

16. The porous hybrid material as claimed in claim 7, wherein the material is a filling and insulating material or as a substitute material for weight reduction.
